(19) United States
(12) Patent Application Publication
(10) Pub. No.: US 2014/0000596 A1
(43) Pub. Date: Jan. 2, 2014

(54) Methods And Apparatus To Zero A Patient Trigger Sensor

(71) Applicants: Jaron Acker, Madison, WI (US); Thomas Kohlmann, McFarland, WI (US)

(72) Inventors: Jaron Acker, Madison, WI (US); Thomas Kohlmann, McFarland, WI (US)

(73) Assignee: INO Therapeutics LLC, Hampton, NJ (US)

(21) Appl. No.: 13/537,146

(22) Filed: Jun. 29, 2012

(51) Int. Cl. A61M 16/12; A61M 16/20

(52) U.S. Cl. 128/203.12

(57) ABSTRACT

Described are methods and apparatus for therapeutic or medical gas delivery that reset or zero the trigger sensor used to detect patient inspiration and expiration. The trigger sensor may be reset automatically during patient expiration to avoid interfering with drug delivery or the breath detection algorithm.

### TECHNICAL FIELD

Embodiments of the present invention generally relate to the field of therapeutic gas administration, particularly to methods and apparatus that zero the trigger sensor used to detect patient inspiration and expiration.

### BACKGROUND

Nitric oxide (NO) is a gas that, when inhaled, acts to dilate blood vessels in the lungs, improving oxygenation of the blood and reducing pulmonary hypertension. Because of this, nitric oxide is provided as a therapeutic gas in the inspiratory breathing gases for patients with pulmonary hypertension.

Some nitric oxide delivery devices administer a pulse of nitric oxide to the patient as the patient inhales spontaneously. Such devices often use a pressure or flow sensor known as a patient trigger sensor to detect when a patient begins inspiration for a particular breath and also to detect each phase of the patients' breath: i.e. inspiratory, expiratory, etc. However, any error in the patient trigger sensor may result in a delayed delivery, a missed dosing opportunity, or an inadvertent dose during the wrong phase of the breath. As the timing of nitric oxide delivery may be critical for some patients, such as within the first half of inspiration, delayed delivery may decrease the effectiveness of nitric oxide therapy. Furthermore, missed breaths or sudden discontinued use of nitric oxide may also have serious consequences, such as rebound hypertension or a decrease in oxygen saturation.

Accordingly, there is a need for new methods and apparatus for providing accurate delivery of therapeutic gases comprising nitric oxide.

### SUMMARY

Provided are methods and apparatus that use one or more trigger zero valves to zero the patient trigger sensor of a therapeutic gas delivery apparatus.

One aspect of the current invention is directed to a therapeutic gas delivery apparatus comprising a therapeutic gas delivery conduit, a passageway in fluid communication with the therapeutic gas delivery conduit, a trigger sensor in fluid communication with the passageway, and one or more trigger zero valves to zero the trigger sensor. The gas source may comprise nitric oxide.

In one or more embodiments of this aspect, the trigger sensor has a first side in fluid communication with the passageway and a second side in fluid communication with a differential pressure port, and the trigger sensor detects a positive or negative pressure differential between the passageway and the differential pressure port. In some embodiments, the delivery apparatus comprises two trigger zero valves, the first trigger zero valve being in fluid communication with the passageway and the first side of the trigger sensor and the second trigger zero valve being in fluid communication with the differential pressure port and the second side of the trigger sensor.

According to one or more embodiments, the first trigger zero valve is a three-way valve having at least two states, the first state enabling fluid communication between the passageway and the first side of the trigger sensor and the second state enabling fluid communication between the first side of the trigger sensor and a pressure source. In some embodiments, the second trigger zero valve is a three-way valve having at least two states, the first state enabling fluid communication between the differential pressure port and the second side of the trigger sensor and the second state enabling fluid communication between the second side of the trigger sensor and the pressure source.

The pressure source may be any suitable pressure source for zeroing the trigger sensor. In some embodiments, the pressure source comprises ambient air. In other embodiments, the pressure source does not contain oxygen. For example, nitric oxide in an inert gas may be used as a pressure source that does not contain oxygen.

In some embodiments, the second state of the first trigger zero valve may prevent fluid communication between the passageway and the pressure source and the second state of the second trigger valve prevents fluid communication between the differential pressure port and the pressure source.

The apparatus may further comprise a control system in communication with the first trigger zero valve and the second trigger zero valve that simultaneously sets the first and second trigger zero valves to their respective second states to make the pressure on the first side of the trigger sensor equal to the pressure on the second side of the trigger sensor. According to one or more embodiments, the control system sets the first and second trigger zero valves to their respective second states during patient expiration. In some embodiments, the control system is in communication with the trigger sensor and sets the first and the second trigger zero valves to their respective second states when the trigger sensor detects a positive pressure differential between the passageway and the differential pressure port.

Some embodiments provide that the differential pressure port may be in fluid communication with ambient air. In other embodiments, the differential pressure port is in fluid communication with a pressurized component of a patient breathing circuit. The pressurized component may comprise a pressurized patient breathing mask.

Another aspect of the present invention pertains to a therapeutic gas delivery apparatus comprising a therapeutic gas delivery conduit, a passageway in fluid communication with the therapeutic gas delivery conduit, a trigger sensor in fluid communication with the passageway, and a trigger zero valve to zero the trigger sensor. The therapeutic gas may comprise nitric oxide.

In some embodiments, the trigger sensor has a first side in fluid communication with the passageway and a second side in fluid communication with a differential pressure port, and the trigger sensor detects a positive or negative pressure differential between the passageway and the differential pressure port. The trigger zero valve may be in fluid communication with the first and second sides of the trigger sensor.

According to one or more embodiments, the trigger zero valve has at least two states, the first state preventing fluid communication between the first and second sides of the trigger sensor and the second state enabling fluid communication between the first and second sides of the trigger sensor. In the second state, the trigger zero valve may also place both sides of the trigger sensor in fluid communication with a pressure source.

The apparatus may further comprise a control system in communication with the trigger zero valve that controls whether the trigger zero valve is in the first state or the second state. In some embodiments, the control system sets the trigger zero valve to the second state during patient expiration. The control system may also be in communication with the trigger sensor and set the trigger zero valve to the second state when the trigger sensor detects a positive pressure differential between the passageway and the differential pressure port.

The pressure source in this aspect may ambient air. Alternatively, in some embodiments, the pressure source does not contain oxygen.

Yet another aspect of the present invention provides a method of administering therapeutic gas, the method comprising sensing inspiration of a patient with a trigger sensor, delivering a pulse of therapeutic gas to the patient during inspiration, and resetting the trigger sensor. The therapeutic gas may comprise nitric oxide. The method may further comprise sensing expiration of the patient and resetting the trigger sensor during patient expiration.

In some embodiments, the trigger sensor is reset after a predetermined period of time since the last trigger sensor reset.

The trigger sensor may be reset in various ways. In some embodiments, resetting the trigger sensor comprises placing a first side and a second side of the trigger sensor in fluid communication with a pressure source such that the pressure on the first side of the trigger sensor is equal to the pressure on the second side of the trigger sensor. Resetting the trigger sensor could also comprise placing a first side of the trigger sensor in fluid communication with a second side of the trigger sensor.

In some embodiments, the trigger sensor is reset automatically without patient intervention. In other embodiments, a user is prompted to reset the trigger sensor.

The foregoing has outlined rather broadly certain features and technical advantages of the present invention. It should be appreciated by those skilled in the art that the specific embodiments disclosed may be readily utilized as a basis for modifying or designing other structures or processes within the scope present invention. It should also be realized by those skilled in the art that such equivalent constructions do not depart from the spirit and scope of the invention as set forth in the appended claims.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a nitric oxide delivery apparatus in accordance with one or more embodiments of the present invention; and

FIG. 2 illustrates a nitric oxide delivery apparatus in accordance with one or more embodiments of the present invention.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Although specific reference is made to nitric oxide delivery apparatuses, it will be understood by a person having ordinary skill in the art that the methods and apparatus described herein may be used to deliver other medical or therapeutic gases. Exemplary gases that may be administered include, but are not limited to, nitric oxide, oxygen, nitrogen, and carbon monoxide. As used herein, the phrase “therapeutic gas” refers to gas used to treat diseases or medical disorders in a patient.

If nitric oxide is used as the therapeutic gas, exemplary diseases or disorders that may be treated include pulmonary arterial hypertension (PAH), chronic obstructive pulmonary disease (COPD), bronchopulmonary dysplasia (BPD), chronic thromboembolic pulmonary hypertension (CTE), idiopathic pulmonary fibrosis (IPF) or pulmonary hypertension (PH), or nitric oxide may be used as an antimicrobial agent.

Provided are methods and apparatus for administering therapeutic gas to a patient that reset or “zero” the patient trigger sensor used to detect patient inspiration and/or expiration. The patient trigger sensor, or breath sensor, is typically calibrated with a two point calibration method, one of the two points being the “zero” pressure reading, i.e. the same pressure on both sides of the differential pressure sensor. The other calibration point is commonly referred to as the “span”, which is a non-zero calibration point. The zero calibration point is typically calibrated more often because inspiratory phase detection is usually monitored for a small pressure difference below zero. By resetting the trigger sensor, the detection of patient inspiration and expiration may be more accurate. If the trigger sensor is not reset, continued use of the gas delivery apparatus may result in the calibration of the trigger sensor being offset, such as by zero drift. Zero drift may occur due to temperature, time, shock or vibration. Such an offset in calibration may lead to false readings, delayed readings or skipped readings for patient inspiration and/or expiration. Any errors in readings may adversely affect the timing of gas administration, which may decrease the efficacy of treatment and may even worsen a patient's condition.

For example, the timing of nitric oxide delivery is critical for disorders such as COPD. For COPD, nitric oxide must be administered in the beginning of inspiration or the patient may experience serious adverse events such as worsened ventilation-perfusion mismatch. Delays as small as tens or hundreds of milliseconds can have a profound effect on the safety and efficacy of nitric oxide treatment. Similarly, for patients with PAH, missed breaths or sudden discontinued use of nitric oxide may result in dangerous rebound hypertension. Therefore, accurate detection of each breath provides an important safety feature.

Accordingly, one aspect of the present invention pertains to a gas delivery apparatus that resets the trigger sensor. The gas delivery apparatus may be a nitric oxide delivery apparatus. In one or more embodiments of this aspect, the gas delivery apparatus comprises a configuration of two or more valves for resetting the trigger sensor.

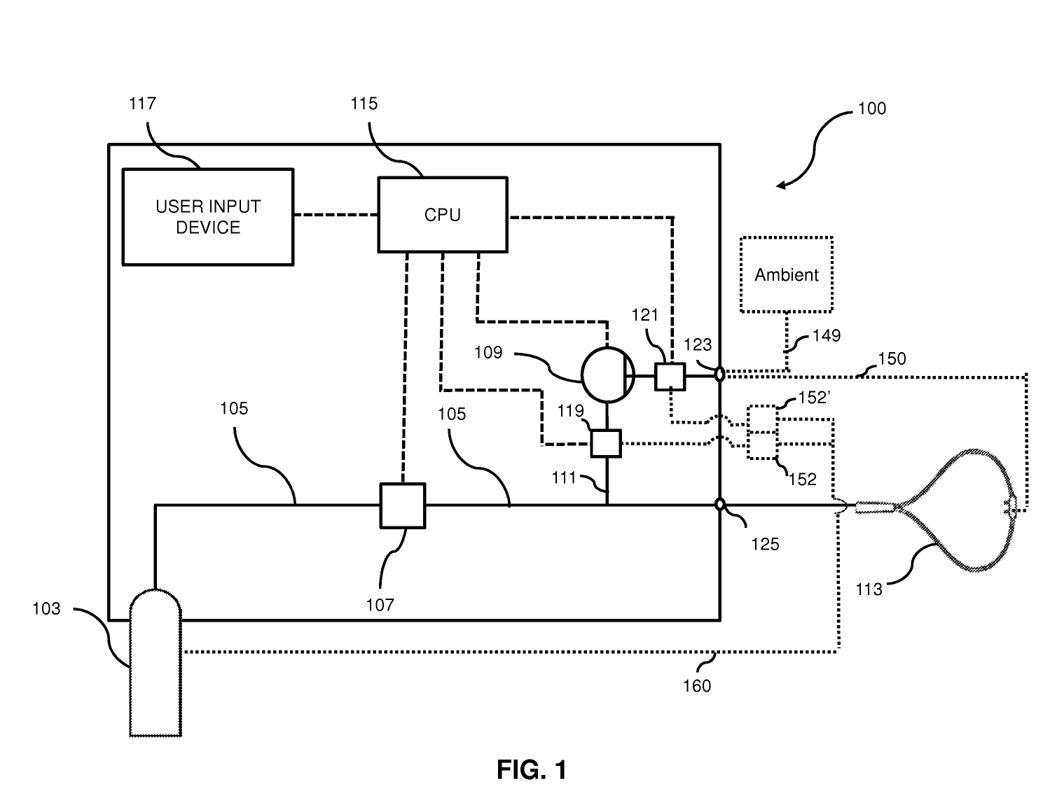

FIG. 1 shows an exemplary nitric oxide delivery apparatus 100 in accordance with this aspect. A source of therapeutic gas containing nitric oxide may include gas storage cylinder 103. Exemplary cylinders may contain NO in a carrier gas such as nitrogen, with a NO concentration ranging from 1 ppm to 20,000 ppm, such as from 5 ppm to 10,000 ppm, or from 10 ppm to 5,000 ppm. In one or more embodiments, the cylinder has a high nitric oxide concentration, such as about 2440 ppm or about 4880 ppm. In other embodiments, the cylinder concentration is about 800 ppm.

Gas storage cylinder 103 is in fluid communication with conduit 105, which carries the therapeutic gas from gas storage cylinder 103 to the gas delivery port 125. The conduit 105 may be in fluid communication with a nasal cannula or other nasal or oral breathing apparatus 113 for delivering the therapeutic gas to the patient. In addition, conduit 105 may comprise a gas hose or tubing section, a pressure regulator, a delivery manifold, etc. Although specific reference is made to nasal cannulas, other types of nasal or oral breathing apparatuses may be used, such as breathing masks. One or more control valves 107 regulate the flow of therapeutic gas through the conduit 105 to the patient. In some embodiments, multiple control valves 107 may be used that provide different flow rates, such as one high flow valve and one low flow valve.

A passageway 111 is in fluid communication with the conduit 105 which connects a patient trigger sensor 109 to the conduit 105. The signal from the trigger sensor 109 may be further processed via hardware and/or software logic by CPU 115, and detects when a patient begins inspiration or expiration, and may provide that information to a control system.

The trigger sensor 109 may be any suitable pressure sensor. In some embodiments, the trigger sensor 109 may be used to determine the patient's inspiration by detecting a negative pressure caused by the patient's breathing effort. This negative pressure may be measured between two reference points, such as between the passageway 111 and the differential pressure port 123. As passageway 111 is in fluid communication with the conduit 105, which in turn is in fluid communication with the patient, the pressure in passageway 111 will drop when a small sub atmospheric pressure in the patient's nose or mouth is created as the patient begins inspiration.

Similarly, the patient trigger sensor 109 may detect the patient's expiration by detecting a positive pressure caused by the patient. In some embodiments, this positive pressure differential is the amount by which the pressure in passageway 111 exceeds the pressure at the differential pressure port 123.

The control system may comprise one or more central processing unit(s) (CPU) 115 in communication with control valve 107 and the patient trigger sensor 109. When the patient trigger sensor 109 determines that a patient is beginning inspiration, the CPU 115 sends a signal to the control valve 107 to open the control valve 107 to deliver a pulse of therapeutic gas. Control valve 107 is only open for a period of time, and the length of the time period, as well as the amount which the control valve 107 opens, will determine the volume of the pulse of therapeutic gas. For example, when control valve 107 is open for a longer period of time, the amount of therapeutic gas in the pulse increases. In certain embodiments, the pulse size may vary from one pulse to the next so that the total amount of therapeutic gas administered over a given time interval is constant, even though a patient's breathing rate may change during this interval. Multiple valves may also be used to deliver the pulse at various flow rates. Alternatively, a proportional valve may be used which allows variable control of flow rate.

Depending on the mode of nitric oxide delivery, the differential pressure port 123 may be at atmospheric pressure, below atmospheric pressure or above atmospheric pressure. If the differential pressure port 123 is open to ambient air, then the pressure at the differential pressure port 123 will be atmospheric pressure. Alternatively, if nitric oxide is delivered into a pressurized patient breathing circuit, such as one that includes a pressurized breathing mask, then the pressure at the differential pressure port 123 may be fluidly connected to the mask or a point within the respiratory device which may be above or below atmospheric pressure.

As shown in FIG. 1, the trigger sensor 109 may have a first side in fluid communication with the passageway 111 and a second side in fluid communication with a differential pressure port 123. A first trigger zero valve 119 may be in fluid communication with the passageway 111 and the first side of the trigger sensor 109. A second trigger zero valve 121 may be in fluid communication with the differential pressure port 123 and the second side of the trigger sensor 109. Both the first trigger zero valve 119 and the second trigger zero valve 121 may be three-way valves.

According to one or more embodiments, the first trigger zero valve 119 is a three-way valve having at least two states. When the first trigger zero valve 119 is in the first state, the first side of the trigger sensor 109 is in fluid communication with the passageway 111. In the second state, the first side of the trigger sensor 109 is in fluid communication with a pressure source. In some embodiments, the second state of the first trigger zero valve 119 prevents fluid communication between the passageway 111 and the pressure source. As used herein, a pressure source is any reservoir or other source of fluid that not appreciably change pressure when placed in fluid communication with a small volume of fluid at a different pressure. In some embodiments, the pressure source is ambient air. In other embodiments, the pressure source does not contain oxygen gas, such as the NO source. As used herein, the phrase “does not contain oxygen gas” means that the pressure source may contain less than 10, 5, 4, 3, 2, 1, 0.5, 0.1 or even 0.05 mole % oxygen gas.

In one or more embodiments, the second trigger zero valve 121 is a three-way valve having at least two states. When the second trigger zero valve 121 is in the first state, the second side of the trigger sensor 109 is in fluid communication with the differential pressure port 123. When the second trigger zero valve 121 is in the second state, the second side of trigger sensor 109 is in fluid communication with a pressure source. This pressure source may be the same or different as the pressure source used for the first trigger zero valve 119. However, in order to reset the trigger sensor 109, the pressure sources used for the first and second trigger zero valves must be at the same pressure. In some embodiments, the second state of the second trigger zero valve 121 prevents fluid communication between the differential pressure port 123 and the pressure source. Some embodiments provide that the pressure source is ambient air.

One problem with NO delivery systems is preventing ambient air from being entrained and mixing with the NO to generate NO₂. Venting the trigger sensor 109 to ambient air using two trigger zero valves or shorting both sides of the trigger zero valve may result in this problem. Accordingly, in some embodiments, the NO delivery apparatus is purged to clear the NO₂ in the system. The NO delivery system may be purged by including a purge valve (not shown) downstream of the trigger zero valve, or by using a 4-way valve as the trigger zero valve 119 or 121.

Alternatively, in some embodiments, a pressure source that does not contain oxygen gas may be used to reset the trigger sensor 109. For example, a source of gas containing NO may be used as the pressure source. The pressurized NO cylinder 103 may be used to supply both sides of the trigger sensor 109 with the same pressure. In some embodiments, the trigger zero valves 119 and 121 may need to be returned to their first state during expiration to prevent additional NO from being pulsed to the patient.

The nitric oxide delivery apparatus 100 may comprise a control system including one or more CPUs 115. The CPU 115 may be in communication with a user input device 117. This user input device 117 can receive desired settings from the user, such as the patient's prescription (in mg/kg ideal body weight, mg/kg/hr, mg/kg/breath, etc.), the patient's age, height, sex, weight, etc.

The CPU 115 may also be in communication with a flow sensor (not shown), which would measure the flow of therapeutic gas through control valve 107. The CPU 115 can be coupled to a memory (not shown) and may be one or more of readily available memory such as random access memory (RAM), read only memory (ROM), flash memory, compact disc, floppy disk, hard disk, or any other form of local or remote digital storage. Support circuits (not shown) can be coupled to the CPU 115 to support the CPU 115, sensors, control valves, etc. in a conventional manner. These circuits include cache, power supplies, clock circuits, input/output circuitry, subsystems, power controllers, signal conditioners, and the like.

The memory may store a set of machine-executable instructions (or algorithms) for calculating the desired volume of the gas pulse and the pulsing schedule to achieve a particular patient prescription. For example, if the patient's breathing rate and the cylinder concentration are known, then the CPU 115 can calculate how much volume of therapeutic gas needs to be administered each breath or set of breaths to provide the desired dosage of nitric oxide. The memory may also record the time that the control valve 107 is open during each pulse, so that future calculations can take into account how much nitric oxide has previously been administered.

The control system may be in communication with the first trigger zero valve 119 and the second trigger zero valve 121, and the control system may control whether each valve is in the first state or second state. The trigger zero valves 119 and 121 may normally be in the first state during nitric oxide delivery, and may only be in the second state when trigger sensor 109 is reset. In some embodiments, the control system simultaneously sets the first trigger zero valve 119 and the second trigger zero valve 121 to their respective second states to make the pressure on the first side of the trigger sensor 109 equal to the pressure on the second side of the trigger sensor 109. When both trigger zero valves are in their second state, the trigger sensor 109 is short-circuited and the trigger sensor 109 may be reset.

The control system may set the trigger zero valves 119 and 121 to their respective second states upon start-up of the nitric oxide delivery apparatus, after the delivery apparatus warms up, and/or on a regular basis. In some embodiments, the trigger sensor 109 may be reset if a predetermined period of time has elapsed since the last reset. For example, the trigger sensor 109 may be reset every hour, day, week, two weeks, or month. It may also be reset more frequently after boot up, starting of therapy, or after a change in atmospheric conditions (i.e. temperature or pressure) is detected.

According to one or more embodiments, the trigger sensor 109 is reset during patient expiration to avoid interfering with nitric oxide delivery or the breath detection algorithm. Thus, the control system may wait until the trigger sensor 109 detects a positive differential between the passageway 111 and the differential pressure port 123 before setting the trigger zero valves to their respective second states.

In one or more embodiments, the trigger sensor 109 is reset automatically, i.e. without patient or other user intervention. In other embodiments, the user is prompted to reset the trigger sensor 109. The trigger sensor 109 may also be reset remotely by a physician at a hospital or at a remote computer interface.

In some embodiments, the memory may store a set of machine-executable instructions (or algorithms), when executed by the CPU 115, cause the apparatus to perform a method comprising: sensing inspiration of a patient with a trigger sensor, delivering a pulse of therapeutic gas containing nitric oxide to the patient during inspiration, and resetting the trigger sensor. The machine-executable instructions may also comprise instructions for any of the other methods described herein.

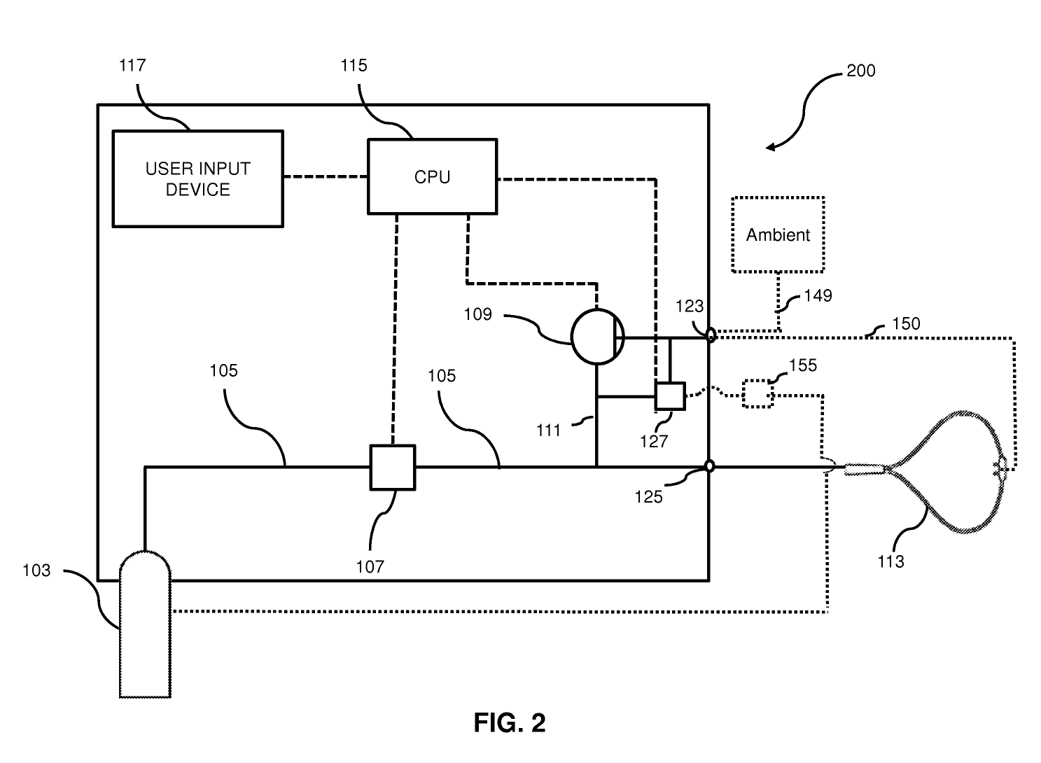

Another aspect of the current invention provides a gas delivery apparatus using a configuration of one or more valves for resetting the trigger sensor. FIG. 2 shows an exemplary nitric oxide delivery apparatus 200 in accordance with one or more embodiments of this aspect. According to one or more embodiments, an apparatus for this aspect may have any of the features described for the first aspect.

Unlike the trigger zero valve configuration in FIG. 1, the apparatus shown in FIG. 2 may have only one trigger zero valve 127. As shown in FIG. 2, the trigger sensor 109 may have a first side in fluid communication with the passageway 111 and a second side in fluid communication with the differential pressure port 123. A trigger zero valve 127 may be in fluid communication with the first and second sides of the trigger sensor 109. In some embodiments, the trigger zero valve 127 may be a valve having at least two states. When the trigger zero valve 127 is in the first state, the first and second sides of the trigger sensor 109 are not in fluid communication. When the trigger zero valve 127 is in the second state, the first side of trigger sensor 109 is in fluid communication with the second side of trigger sensor 109.

In one or more embodiments, the trigger zero valve 127 may a three-way valve. In these embodiments, the three-way valve may have a state that places the first and second sides of the trigger sensor 109 in fluid communication with a pressure source. According to some embodiments, the pressure source is ambient air. In other embodiments, the pressure source does not contain oxygen gas.

As with trigger zero valves 119 and 121, trigger zero valve 127 may be in communication with a control system that determines whether trigger zero valve 127 is in the first or second state. The trigger zero valve 127 may normally be in the first state during nitric oxide delivery, and may only be in the second state when trigger sensor 109 is reset. When trigger zero valve 127 is in the second state, the pressure on the first side of trigger sensor 109 is equal to the pressure on the second side of trigger sensor 109, and trigger sensor 109 is reset. In some embodiments, the trigger sensor 109 is reset during patient expiration. Thus, the control system may wait until the trigger sensor 109 detects a positive pressure differential before resetting the trigger sensor 109.

Another aspect of the current invention provides a method of administering a therapeutic or medical gas, the method comprising sensing inspiration of a patient with a trigger sensor, delivering a pulse of therapeutic or medical gas, and resetting the trigger sensor. The therapeutic or medical gas may be nitric oxide.

In some embodiments of this aspect, the trigger sensor is reset during patient expiration, so as to avoid interfering with breath detection or drug delivery. Patient expiration may be detected by using the same or different trigger sensor.

The trigger sensor may be reset upon start-up of the gas delivery apparatus, after the delivery apparatus warms up, and/or on a regular basis. In some embodiments, the trigger sensor may be reset if a predetermined period of time has elapsed since the last reset. For example, the trigger sensor may be reset every hour, day, week, two weeks, or month.

The trigger sensor may be reset in any manner described herein. According to one or more embodiments, resetting the trigger sensor comprises placing a first side and a second side of the trigger sensor in fluid communication with one or more pressure sources. This may make the pressure on the first side of the trigger sensor equal to the pressure on the second side of the trigger sensor. In some embodiments, the pressure source is ambient air. In other embodiments, the pressure source does not contain oxygen.

Some embodiments provide that resetting the trigger sensor comprises placing the first side of the trigger sensor in fluid communication with the second side of the trigger sensor, such as by using the configuration comprising at least one valve shown in FIG. 2.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A therapeutic gas delivery apparatus comprising:
a therapeutic gas delivery conduit in fluid communication with a nasal cannula or breathing apparatus to deliver a therapeutic gas to a patient when the delivery conduit is connected to a gas source;
a passageway in fluid communication with the nasal cannula or breathing apparatus;
a trigger sensor having a first side in fluid communication with the passageway and a second side in fluid communication with a differential pressure port in fluid communication with ambient air or a pressurized patient breathing mask, wherein the trigger sensor detects a positive or negative pressure differential between the passageway and the differential pressure port, to detect patient inspiration and expiration;
a first trigger zero valve in fluid communication with the passageway and the first side of the trigger sensor;
a second trigger zero valve in fluid communication with the differential pressure port and the second side of the trigger sensor;
a control system in communication with the first trigger zero valve and the second trigger zero valve that resets the trigger sensor during patient expiration.

2. The apparatus of claim 1, wherein the first trigger zero valve is a three-way valve having at least a first state and a second state, the first state enabling fluid communication between the passageway and the first side of the trigger sensor and the second state enabling fluid communication between the first side of the trigger sensor and a pressure source, and wherein the second trigger zero valve is a three-way valve having at least a first state and a second state, the first state enabling fluid communication between the differential pressure port and the second side of the trigger sensor and the second state enabling fluid communication between the second side of the trigger sensor and the pressure source.

3. The apparatus of claim 2, wherein the pressure source is ambient air.

4. The apparatus of claim 2, wherein the pressure source does not contain oxygen.

5. The apparatus of claim 2, wherein the second state of the first trigger zero valve prevents fluid communication between the passageway and the pressure source and the second state of the second trigger valve prevents fluid communication between the differential pressure port and the pressure source.

6. The apparatus of claim 2, wherein the control system, in communication with the first trigger zero valve and the second trigger zero valve, simultaneously sets the first and second trigger zero valves to their respective second states to make the pressure on the first side of the trigger sensor equal to the pressure on the second side of the trigger sensor.

7. The apparatus of claim 6, wherein the control system sets the first and second trigger zero valves to their respective second states during patient expiration.

8. The apparatus of claim 6, wherein the control system, in communication with the trigger sensor, sets the first and the second trigger zero valves to their respective second states when the trigger sensor detects a positive pressure differential between the passageway and the differential pressure port.

9. (canceled)

10. (canceled)

11. (canceled)

12. The apparatus of claim 1, wherein the gas source comprises nitric oxide.

13. A therapeutic gas delivery apparatus comprising:
a therapeutic gas delivery conduit in fluid communication with a nasal cannula or breathing apparatus and to deliver a therapeutic gas to a patient when the delivery conduit is connected to a gas source;
a passageway in fluid communication with the nasal cannula or breathing apparatus;
a trigger sensor having a first side in fluid communication with the passageway and a second side in fluid communication with a differential pressure port in fluid communication with ambient air or a pressurized patient breathing mask, wherein the trigger sensor detects a positive or negative pressure differential between the passageway and the differential pressure port;
a trigger zero valve in fluid communication with the first and second sides of the trigger sensor; and
a control system in communication with the trigger zero valve that resets the trigger sensor during patient expiration.

14. The apparatus of claim 13, wherein the trigger zero valve has at least a first state and a second state, the first state preventing fluid communication between the first and second sides of the trigger sensor and the second state enabling fluid communication between the first and second sides of the trigger sensor.

15. The apparatus of claim 14, wherein the control system, in communication with the trigger zero valve, controls whether the trigger zero valve is in the first state or the second state.

16. The apparatus of claim 15, wherein the control system sets the trigger zero valve to the second state during patient expiration.

17. The apparatus of claim 15, wherein the control system, in communication with the trigger sensor, sets the trigger zero valve to the second state when the trigger sensor detects a positive pressure differential between the passageway and the differential pressure port.

18. The apparatus of claim 14, wherein when the trigger zero valve is in the second state, the first and second sides of the trigger sensor are in fluid communication with a pressure source.

19. The apparatus of claim 13, wherein the gas source comprises nitric oxide.

20. A method of administering therapeutic gas, the method comprising:
sensing inspiration of a patient using a trigger sensor detecting a pressure differential applied on the trigger sensor relative to ambient air or a pressurized patient breathing mask;
during patient inspiration, delivering a pulse of therapeutic gas to the patient with one or more control valves; and
resetting the trigger sensor with one or more trigger zero valves during patient expiration.

21. The method of claim 20, further comprising sensing expiration of the patient, and wherein the trigger sensor is reset during patient expiration.

22. The method of claim 20, wherein the trigger sensor is reset after a predetermined period of time since the last trigger sensor reset.

23. The method of claim 20, wherein resetting the trigger sensor comprises placing the one or more trigger zero valves in a state such that a first side and a second side of the trigger sensor are each in fluid communication with a pressure source so that the pressure on the first side of the trigger sensor is equal to the pressure on the second side of the trigger sensor.

24. The method of claim 20, wherein resetting the trigger sensor comprises placing a first side of the trigger sensor in fluid communication with a second side of the trigger sensor.

25. The method of claim 20, wherein the therapeutic gas comprises nitric oxide.

26. The method of claim 20, wherein the trigger sensor is reset automatically without patient intervention.

27. The apparatus of claim 1, wherein the therapeutic gas delivery conduit is in direct fluid communication with the nasal cannula or breathing apparatus and the passageway.

28. The apparatus of claim 1, further comprising:
a one or more control valves regulating flow of the therapeutic gas to the nasal cannula or breathing apparatus, the one or more control valves being in fluid communication with the therapeutic gas delivery conduit, the gas source, and the nasal cannula or breathing apparatus; and
wherein the one or more control valves is located between the gas source and the nasal cannula or breathing apparatus.

29. The apparatus of claim 28, wherein the nasal cannula or breathing apparatus is in fluid communication with the one or more control valves at a first offset distance and the trigger sensor at a second offset distance, the first offset distance being further from the nasal cannula or breathing apparatus than the second offset distance.

30. The apparatus of claim 1, wherein the control system, in communication with the trigger sensor and a one or more control valves, opens the one or more control valves during patient inspiration.

31. The apparatus of claim 13, wherein the therapeutic gas delivery conduit is in direct fluid communication with the nasal cannula or breathing apparatus and the passageway.

32. The apparatus of claim 13, further comprising:
a one or more control valves regulating flow of the therapeutic gas to the nasal cannula or breathing apparatus, the one or more control valves being in fluid communication with the therapeutic gas delivery conduit, the gas source, and the nasal cannula or breathing apparatus; and
wherein the one or more control valves is located between the gas source and the nasal cannula or breathing apparatus.

33. The apparatus of claim 32, wherein the nasal cannula or breathing apparatus is in fluid communication with the one or more control valves at a first offset distance and the trigger sensor at a second offset distance, the first offset distance being further from the nasal cannula or breathing apparatus than the second offset distance.

34. The apparatus of claim 1, wherein the control system, in communication with the trigger sensor and a one or more control valves, opens the one or more control valves during patient inspiration.
